(19) United States
(12) Patent Application Publication
(10) Pub. No.: US 2021/0371440 A1
(43) Pub. Date: Dec. 2, 2021

(54) TLR7 AND / OR TLR8 AGONISTS

(71) Applicant: GLAXOSMITHKLINE BIOLOGICALS SA, Rixensart (BE)

(72) Inventor: Helene BAZIN-LEE, Hamilton, MT (US)

(73) Assignee: GLAXOSMITHKLINE BIOLOGICALS SA, Rixensart (BE)

(21) Appl. No.: 17/043,549

(22) PCT Filed: Apr. 12, 2019
(86) PCT No.: PCT/EP2019/059388

§ 371 Date: Sep. 29, 2020

Related U.S. Application Data
(60) Provisional application No. 62/657,084, filed on Apr. 13, 2018.

(51) Int. Cl. C07F 9/6561; A61K 39/39; C07D 473/18

(52) U.S. Cl. CPC C07F 9/65616; A61K 2039/55555; C07D 473/18; A61K 39/39

(57) ABSTRACT

The invention relates to a novel lipidated oxoadenine compound of formula (I) and its use as a vaccine adjuvant and as a TLR7 and/or TLR8 agonist.

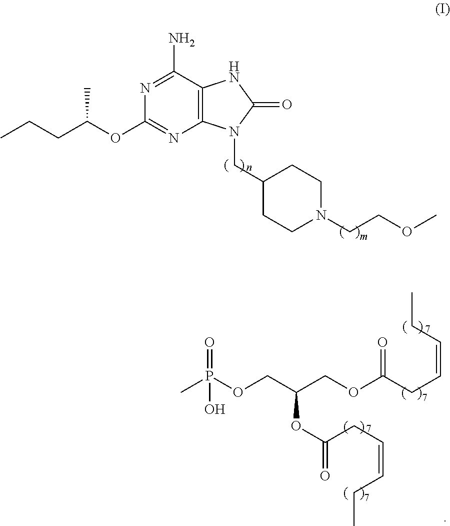

### GOVERNMENT SUPPORT CLAUSE

Aspects of this invention were made with United States government support under Contract No. HHSN272200900036C awarded by the National Institutes of Health (NIH). The United States government may have certain rights in the invention.

### FIELD OF THE INVENTION

The invention relates to novel lipidated oxoadenines and their use as vaccine adjuvants and as TLR7 and/or TLR8 agonists. Further provided are methods of production of said compounds.

### BACKGROUND OF THE INVENTION

Adjuvants are included in vaccines to improve humoral and cellular immune responses, particularly in the case of poorly immunogenic subunit vaccines. Similar to natural infections by pathogens, adjuvants rely on the activation of the innate immune system to promote long-lasting adaptive immunity. As simultaneous activation of multiple innate immune pathways is a feature of natural infections, adjuvants may combine multiple immunostimulants in order to promote adaptive immune responses to vaccination.

Toll-like receptor (TLR) 7 and TLR8 play an important role in the immune response to viral infection. A few different classes of small molecule mimetics of the natural (U- and/or G-rich) viral ssRNA ligands have been identified for TLR7 and TLR8 activation. These include certain antiviral compounds related to oxidized guano sine metabolites (oxoguanosines), which primarily interact with TLR7 (Heil, F, 2003; Hemmi, 2002) and derivatives of adenine which engage TLR7 and/or TLR8. The immune stimulating ability of these compounds has been attributed to the TLR/MyD88-dependent signaling pathways and the production of cytokines, including IL-6 and type I (particularly interferon-alpha) and type II interferons. TLR7 or TLR8 activation leads to the upregulation of co-stimulatory molecules (e.g. CD-40, CD-80, CD-86) and class I and II MHC molecules on dendritic cells (DCs). DCs are the principal cells of the immune system involved in uptake and presentation of antigens to T lymphocytes. Plasmacytoid dendritic cells (pDCs), which preferentially express TLR7, are professional interferon-a producing cells; whereas myeloid dendritic cells (mDCs) express TLR8. TLR8 activation on mDCs leads to the preferential production of pro-inflammatory cytokines such as IL-12, TNF-a, and IFN-γ and cell-mediated immunity (CMI).

WO2010/018134 discloses oxoadenine TLR7 and/or TLR8 agonists of use as adjuvants.

WO2011/017611 discloses lipidated oxoadenine TLR7 and/or TLR8 agonists of use as adjuvants.

WO2016/142880 discloses oxoadenine compounds and their covalent linking to antigens.

WO2017/102652 discloses peg lipidated imidazoquinoline TLR7 and/or TLR8 agonists.

Bazin et al (2015) discloses oxoadenine TLR7 and/or TLR8 agonists.

Smith et al (2016) discloses imidazoquinoline and oxoadenine TLR7 and/or TLR8 agonists of use as adjuvants.

There remains a need for other immunostimulatory compounds demonstrating different or improved activity profiles or other pharmacological or practical benefits.

### SUMMARY OF THE INVENTION

The present invention provides a compound of formula (I):

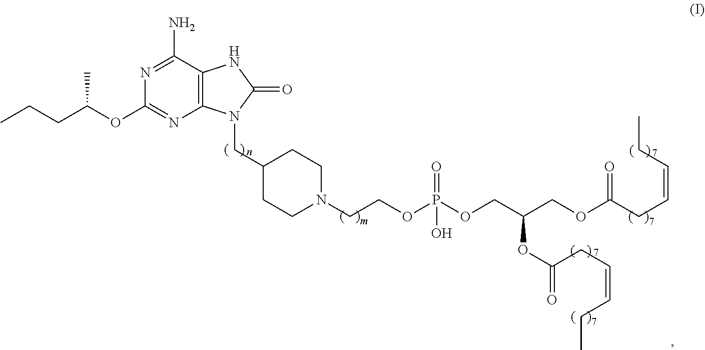

wherein n is 1, 2, 3, 4, or 5 and m is 0, 1, 2, 3, or 4.

There is also provided a compound of formula (I):

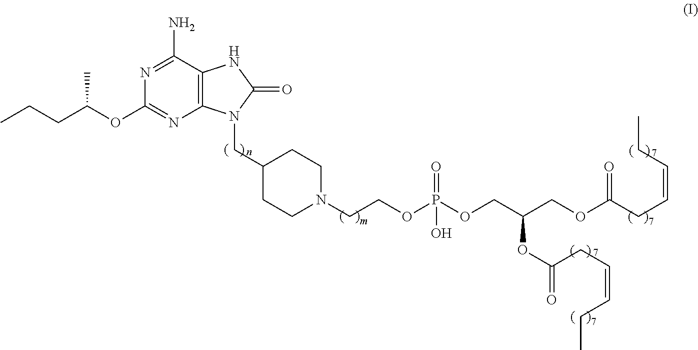

wherein n is 1, 2, 3, 4, or 5 and m is 0, 1, 2, 3, or 4, for use as a TLR7 and/or TLR8 agonist.

Additionally provided is a compound of formula (I):

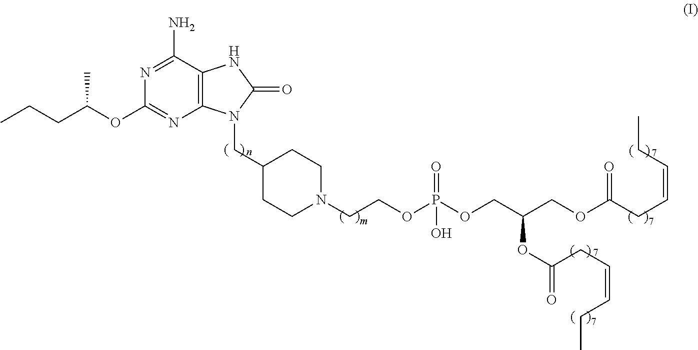

wherein n is 1, 2, 3, 4, or 5 and m is 0, 1, 2, 3, or 4, for use as an adjuvant.

Also provided is the use of a compound of formula (I):

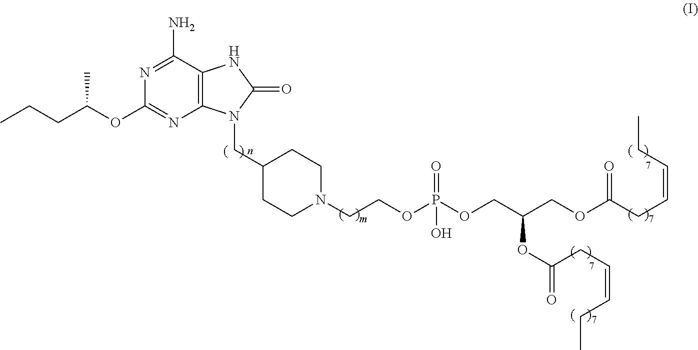

wherein n is 1, 2, 3, 4, or 5 and m is 0, 1, 2, 3, or 4, in the manufacture of a medicament for use as a TLR7 and/or TLR8 agonist.

There is also provided the use of a compound of formula (I):

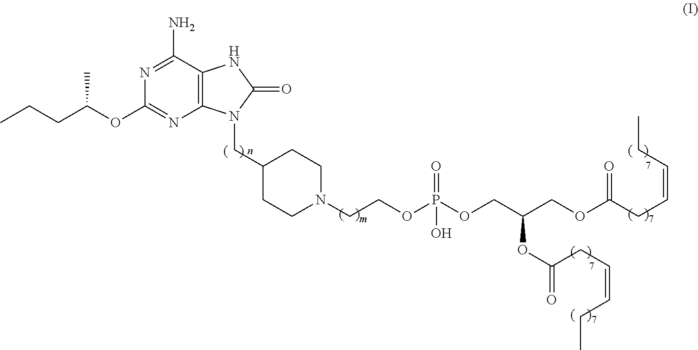

wherein n is 1, 2, 3, 4, or 5 and m is 0, 1, 2, 3, or 4, in the manufacture of an adjuvant.

The present invention provides a method for eliciting an immune response in a subject, said method comprising the step of administering a compound of formula (I):

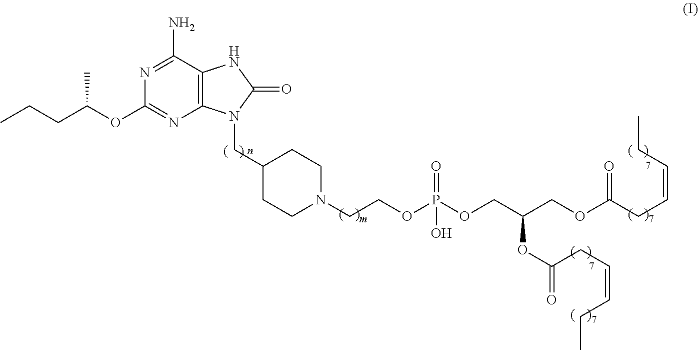

wherein n is 1, 2, 3, 4, or 5 and m is 0, 1, 2, 3, or 4, to the subject.

Also provided is a method for adjuvanting an immune response in a subject, said method comprising the step of administering a compound of formula (I):

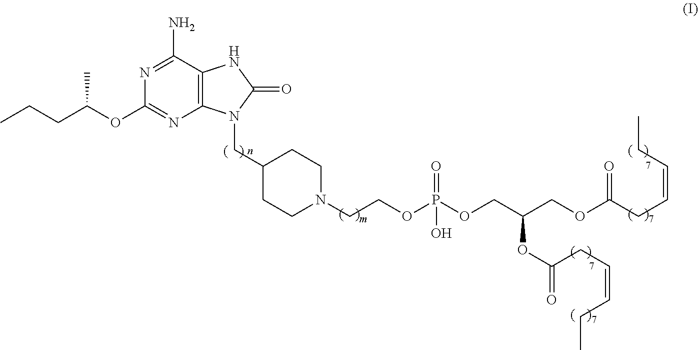

wherein n is 1, 2, 3, 4, or 5 and m is 0, 1, 2, 3, or 4, to the subject.

A further aspect of the invention is a compound of formula (I)

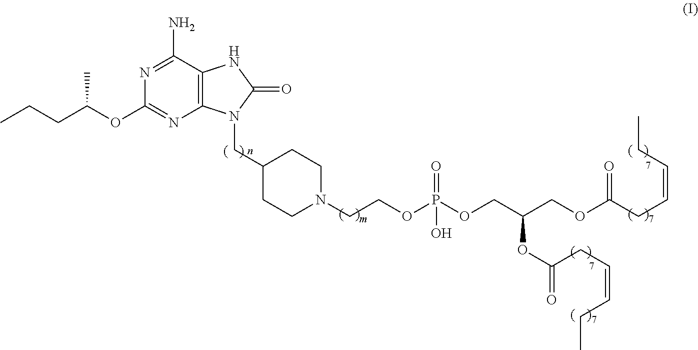

wherein n is 1, 2, 3, 4, or 5 and m is 0, 1, 2, 3, or 4, for use as a medicament.

A compound of formula (I) may be provided in the form of a salt and/or solvate thereof and/or prodrug thereof. In one embodiment, the compound of formula (I) is provided in the form of a pharmaceutically acceptable salt.

### DETAILED DESCRIPTION OF THE INVENTION

There is provided a compound of formula (I):

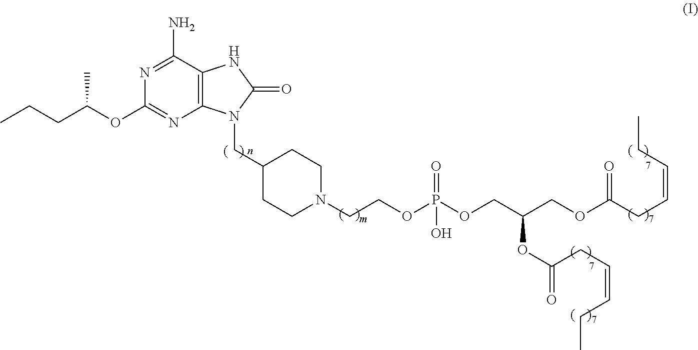

wherein n is 1, 2, 3, 4, or 5 and m is 0, 1, 2, 3, or 4, or a pharmaceutically acceptable salt and/or solvate and/or prodrug thereof.

There is also provided a compound of formula (I):

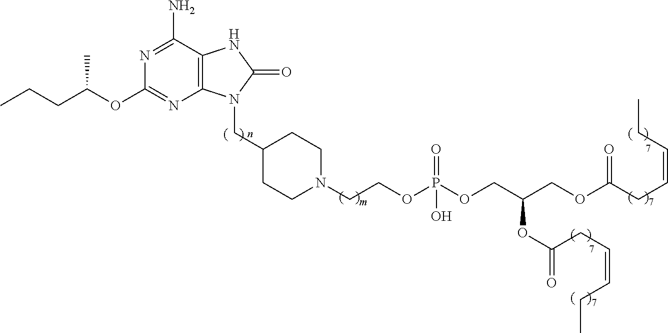

wherein n is 1, 2, 3, 4, or 5 and m is 0, 1, 2, 3, or 4, or a pharmaceutically acceptable salt and/or solvate and/or prodrug thereof for use in eliciting an immune response.

Additionally provided a compound of formula (I):

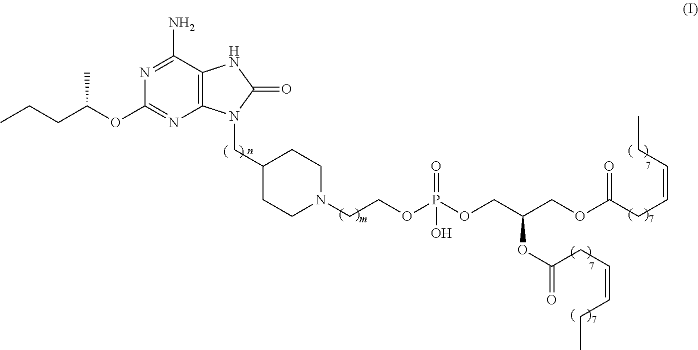

wherein n is 1, 2, 3, 4, or 5 and m is 0, 1, 2, 3, or 4, or a pharmaceutically acceptable salt and/or solvate and/or prodrug thereof, for use as an adjuvant.

Additionally provided a compound of formula (I):

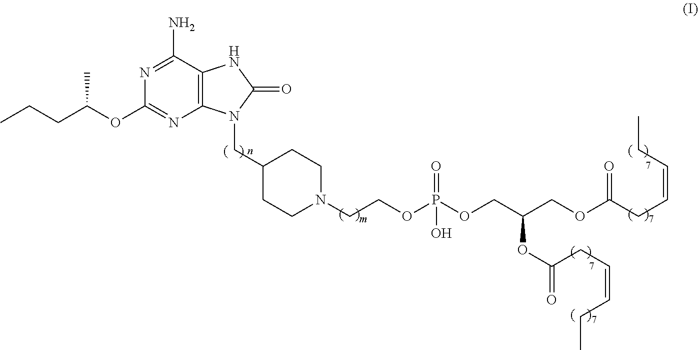

wherein n is 1, 2, 3, 4, or 5 and m is 0, 1, 2, 3, or 4, or a pharmaceutically acceptable salt and/or solvate and/or prodrug thereof, for use as a TLR7 and/or TLR8 agonist.

Also provided is the use of a compound of formula (I):

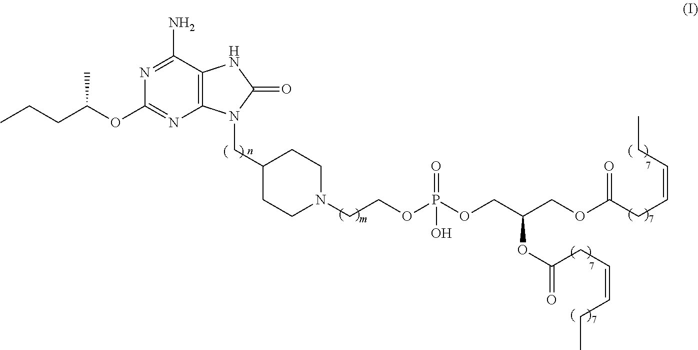

wherein n is 1, 2, 3, 4, or 5 and m is 0, 1, 2, 3, or 4, or a pharmaceutically acceptable salt and/or solvate and/or prodrug thereof, in the manufacture of a medicament for eliciting an immune response.

There is also provided the use of a compound of formula (I):

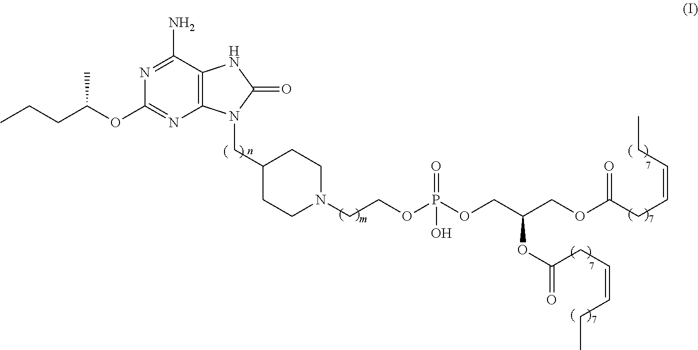

wherein n is 1, 2, 3, 4, or 5 and m is 0, 1, 2, 3, or 4, or a pharmaceutically acceptable salt and/or solvate and/or prodrug thereof, in the manufacture of an adjuvant.

There is also provided the use of a compound of formula (I):

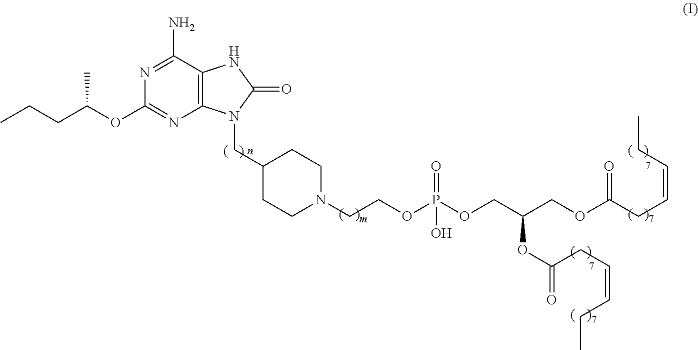

wherein n is 1, 2, 3, 4, or 5 and m is 0, 1, 2, 3, or 4, or a pharmaceutically acceptable salt and/or solvate and/or prodrug thereof, in the manufacture of a medicament for use as TLR7 and/or TLR8 agonist.

The present invention provides a method for eliciting an immune response in a subject, said method comprising the step of administering a compound of formula (I):

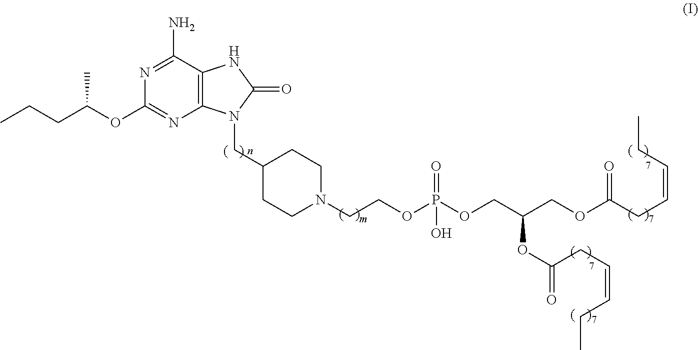

wherein n is 1, 2, 3, 4, or 5 and m is 0, 1, 2, 3, or 4, or a pharmaceutically acceptable salt and/or solvate and/or prodrug thereof, to the subject.

Also provided is a method of adjuvanting an immune response in a subject, said method comprising the step of administering a compound of formula (I):

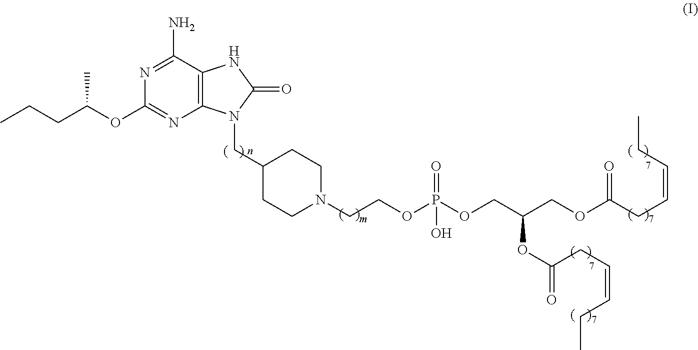

wherein n is 1, 2, 3, 4, or 5 and m is 0, 1, 2, 3, or 4, or a pharmaceutically acceptable salt and/or solvate and/or prodrug thereof, to the subject.

A further aspect of the invention is a compound of formula (I)

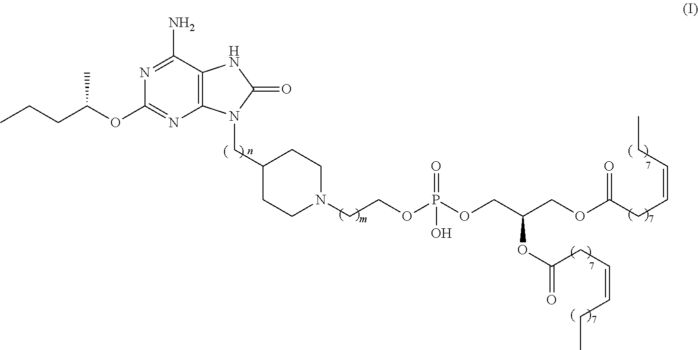

wherein n is 1, 2, 3, 4, or 5 and m is 0, 1, 2, 3, or 4, or a pharmaceutically acceptable salt and/or solvate thereof and/or prodrug thereof for use as a medicament.

In one embodiment n is 1 (i.e. a forming a methylene linker). In a second embodiment n is 2 (i.e. a forming an ethylene linker). In a third embodiment n is 3 (i.e. a forming a propylene linker). In a fourth embodiment n is 4 (i.e. a forming a butylene linker). In a fifth embodiment n is 5 (i.e. a forming a pentylene linker).

In one embodiment m is 0 (i.e. a forming a methylene linker). In a second embodiment m is 1 (i.e. a forming an ethylene linker). In a third embodiment m is 2 (i.e. a forming a propylene linker). In a fourth embodiment m is 3 (i.e. a forming a butylene linker). In a fifth embodiment m is 4 (i.e. a forming a pentylene linker).

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The compound of formula (I), or the pharmaceutically salt and/or solvate and/or prodrug thereof, may be provided together with at least one other therapeutically active agent e.g. more than one compound of formula (I) may be provided, or one compound of formula (I) with a further agent which is not a compound of formula (I).

There is also provided a process for preparing a pharmaceutical composition which comprises admixing a compound of formula (I), or a pharmaceutically acceptable salt and/or solvate and/or prodrug thereof, with one or more pharmaceutically acceptable diluents or carriers.

Formulation of pharmaceutically-acceptable excipients and carrier solutions is well-known to those of skill in the art, as is the development of suitable dosing and treatment regimens for using the particular compositions described herein in a variety of treatment regimens. The compound of formula (I) or its pharmaceutically acceptable salts and/or solvates and/or prodrugs thereof may be administered by any convenient method, e.g. oral, parenteral, intravenous, buccal, sublingual, rectal, transdermal, intradermal, intranasal, intratympanic, intracochlear or intramuscular administration. Other routes of administration include via the mucosal surfaces.

Typically a compound of formula (I), or a pharmaceutically acceptable salt and/or solvate and/or prodrug thereof, is administered parenterally, such as intramuscularly or subcutaneously.

Salts of a compound of formula (I) include pharmaceutically acceptable salts and salts which may not be pharmaceutically acceptable but may be useful in the preparation of a compound of formula (I) and pharmaceutically acceptable salts thereof. Salts may be derived from certain inorganic or organic acids, or certain inorganic or organic bases. For a review on suitable salts see for example Berge et al (1977).

Solvates of a compound of formula (I) include pharmaceutically acceptable solvates and solvates which may not be pharmaceutically acceptable but may be useful in the preparation of a compound of formula (I) and pharmaceutically acceptable solvates thereof. Pharmaceutically acceptable solvates include hydrates.

Salts and hydrates may be provided in stoichiometric and non-stoichiometric amounts.

Consequently, there is provided a compound of formula (I):

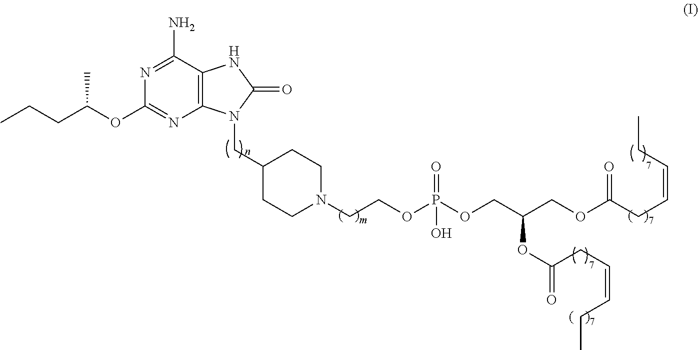

wherein n is 1, 2, 3, 4, or 5 and m is 0, 1, 2, 3, or 4, or a salt and/or solvate thereof.

An “immune response” associated with TLR7 and/or TLR8 is an immune response which involves activation of TLR7 and/or TLR8 receptors. Activation of TLR7 and/or TLR8 receptors may be determined in vitro using methods such as those described in the Examples.

As used herein, a “subject” is any mammal, including but not limited to humans, non-human primates, farm animals such as cattle, sheep, pigs, goats and horses; domestic animals such as cats, dogs, rabbits; laboratory animals such as mice, rats and guinea pigs that exhibit at least one symptom associated with a disease, have been diagnosed with a disease, or are at risk for developing a disease. The term does not denote a particular age or sex. Suitably the subject is a human.

Isotopically-labelled compounds which are identical to those recited in formula (I) but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”) are also contemplated for the uses and method of the invention. Examples of isotopes that can be incorporated into the compounds of the invention include isotopes of hydrogen, carbon, nitrogen, oxygen, fluorine, iodine and chlorine such as ²H (deuterium), ³H, ¹¹C, ¹³C, ¹⁴C, ¹⁸F, ¹²³I or ¹²⁵I, which may be naturally occurring or non-naturally occurring isotopes.

A compound of formula (I), or a salt and/or solvate and/or prodrug of said compound, that contains the aforementioned isotopes and/or other isotopes of other atoms are contemplated for use for the uses and method of the present invention. Isotopically labelled compounds of the present invention, for example those into which radioactive isotopes such as ³H or ¹⁴C have been incorporated, are useful in drug and/or substrate tissue distribution assays. Tritiated, i.e. ³H, and carbon-14, i.e. ¹⁴C, isotopes are particularly preferred for their ease of preparation and detectability. ¹¹C and ¹⁸F isotopes are particularly useful in PET (positron emission tomography).

Since a compound of formula (I), or a pharmaceutically acceptable salt and/or solvate and/or prodrug thereof, is intended for use in pharmaceutical compositions it will readily be understood that it is preferably provided in substantially pure form, for example at least 60% pure, more suitably at least 75% pure and preferably at least 85%, especially at least 98% pure (% are on a weight for weight basis). Impure preparations of a compound may be used for preparing the more pure form used in the pharmaceutical compositions.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

The composition may contain from 0.001% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.001 mg to 10 mg, for example from 0.01 mg to 1 mg, of the active material, depending on the method of administration.

In one aspect of the invention, the compound of formula (I) or a salt and/or prodrug of said compound, is provided in the form of an aqueous adjuvant composition.

When formulated, a compound of formula (I), or a pharmaceutically acceptable salt and/or solvate and/or prodrug thereof, may be provided with a suitable carrier such as liposomes. The term ‘liposome’ is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds. Liposome size may vary from 30 nm to several um depending on the phospholipid composition and the method used for their preparation. In particular embodiments of the invention, the liposome size will be in the range of 50 nm to 500 nm and in further embodiments 50 nm to 200 nm. Optimally, the liposomes should be stable and have a diameter of ˜100 nm to allow sterilization by filtration.

Suitably, the compositions used in the present invention have a human dose volume of between 0.05 ml and 1 ml, such as between 0.1 and 0.5 ml, in particular a dose volume of about 0.5 ml, or 0.7 ml. The volumes of the compositions used may depend on the delivery route and location, with smaller doses being given by the intradermal route.

The pH of a liquid preparation is adjusted in view of the components of the composition and necessary suitability for administration to the subject. Suitably, the pH of a liquid mixture is at least 4, at least 5, at least 5.5, at least 5.8, at least 6. The pH of the liquid mixture may be less than 9, less than 8, less than 7.5 or less than 7. In other embodiments, pH of the liquid mixture is between 4 and 9, between 5 and 8, such as between 5.5 and 8. In a further embodiment, a buffer is added to the composition.

It is well known that for parenteral administration solutions should have a pharmaceutically acceptable osmolality to avoid cell distortion or lysis. A pharmaceutically acceptable osmolality will generally mean that solutions will have an osmolality which is approximately isotonic or mildly hypertonic. Suitably the compositions of the present invention when reconstituted will have an osmolality in the range of 250 to 750 mOsm/kg, for example, the osmolality may be in the range of 250 to 550 mOsm/kg, such as in the range of 280 to 500 mOsm/kg.

Osmolality may be measured according to techniques known in the art, such as by the use of a commercially available osmometer, for example the Advanced® Model 2020 available from Advanced Instruments Inc. (USA).

A desired osmolality may be achieved by the inclusion of salts or through the use of non-ionic isotonicity agents. In one embodiment of the present invention, suitable non-ionic isotonicity agents are polyols, sugars (in particular sucrose, fructose, dextrose or glucose) or amino acids such as glycine. In one embodiment the polyol is a sugar alcohol especially a C3-6 sugar alcohol. Exemplary sugar alcohols include glycerol, erythritol, threitol, arabitol, xylitol, ribitol, sorbitol, mannitol, dulcitol and iditol. In a specific example of this embodiment, a suitable non-ionic isotonicity agent is sorbitol. In a particular embodiment of the invention the non-ionic isotonicity agent in the compositions of the invention is sucrose and/or sorbitol.

Parenteral compositions are suitably sterile.

The compound of formula (I), or the salt and/or solvate and/or prodrug thereof, may be formulated as a pharmaceutical composition with one or more pharmaceutically acceptable diluents or carriers.

The compound of formula (I), or the salt and/or solvate and/or prodrug thereof, may be formulated as an immunogenic composition, together with at least one immunogen or antigen.

The invention also provides a kit comprising:
(i) a first composition comprising at least one compound of formula (I), or the salt and/or solvate and/or prodrug thereof; and
(ii) a second composition comprising at least one immunogen or antigen.

In some embodiments the compositions, kits and methods of the present invention may include a polynucleotide encoding the immunogen or antigen.

By the term immunogen is meant a polypeptide which is capable of eliciting an immune response. Suitably the immunogen is an antigen which comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

The antigen may be derived (such as obtained from) from a human or non-human pathogen including, e.g., bacteria, virus, fungi, parasitic microorganisms or multicellular parasites which infect human and non-human vertebrates, or from a cancer cell or tumor cell. The antigen is suitably derived (such as obtained from) from a human pathogen, in particular a human pathogen selected from the group consisting of bacteria, virus, fungi, parasitic microorganisms and multicellular parasites.

The compound of formula (I) may be prepared as described in the Examples section.

Accordingly, there is provided a process for the preparation of a compound of formula (I) which process comprises reacting a compound of formula (II):

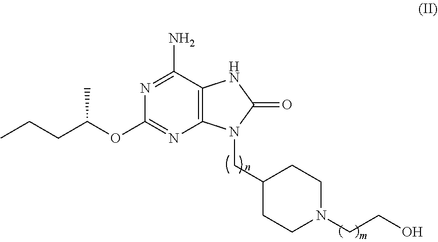

with a compound of formula (V):

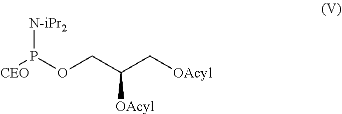

where acyl is oleoyl, in the presence of a solvent such as acetonitrile, followed by an oxidisation step of the phosphite and a de-protecting step. Other suitably protected derivatives of the compound of formula (V) may also be utilised in place of the iPr and CE functions.

Compounds of formula (II) have utility in the manufacture of the lipidated compounds of the invention but may also have TLR7 and/or TLR activity and form an aspect of the present invention.

By the term prodrug is meant herein a derivative of the compound of formula (I) which is metabolised in the body to produce the compound of formula (I).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The invention will be further described by reference to the following, non-limiting examples.

### EXAMPLES

Synthesis of Compound 1 (n=1, m=1)

Oxoadenines and methods for preparing oxoadenines are known in the art and in particular are disclosed in WO2010/018134. Analogous methods were used to synthesise the intermediate of formula (II):

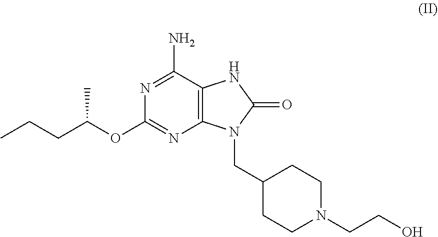 [SMILES: CCC[C@H](C)Oc1nc(N)c2[nH]c(=O)n(CC3CCN(CCO)CC3)c2n1]

The scheme below gives an outline of the steps for its synthesis:

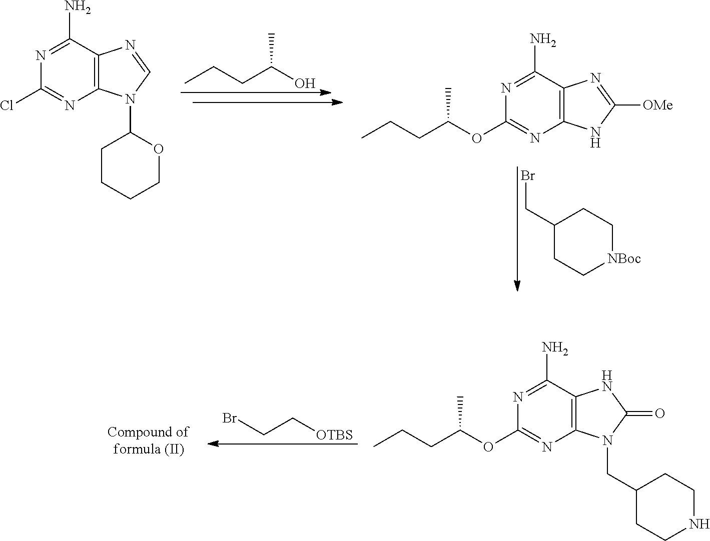

Phosphatidylation of the oxoadenine intermediate of formula (II) using the method shown in the scheme below was completed to afford Compound 1 (97% purity) in 42% overall yield:

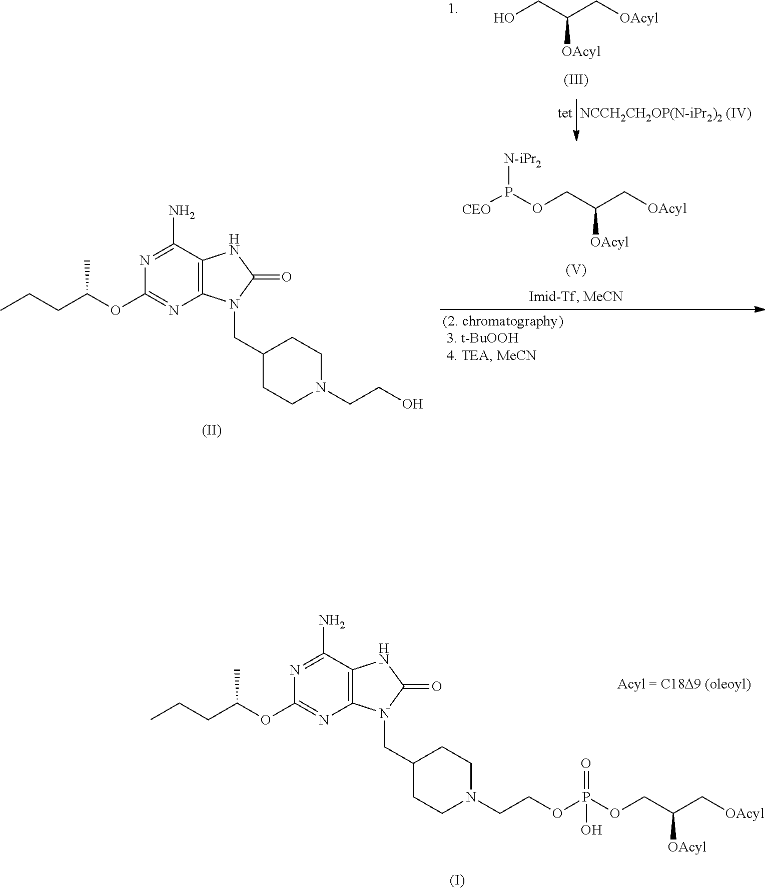

Compound 1 was prepared by:
a) reacting a compound of formula (III) (prepared according to methods known in the art, Tashiro 2013; Modica 2012; Gege 2000), with a phosphordiamidite reagent of formula (IV) (commercially available) according to methods known in the art;
b) reacting a compound of formula (V) (not isolated) in-situ with the oxoadenine compound of formula (II) (Gerster 2005; Izumi 2003) according to methods known in the art;
c) oxidizing the resulting compound from step (b) and removing the protecting group according to methods known in the art to produce a compound of formula (I).

General conditions which may be used are as follows:

A compound of formula (III) (2.0 eq) and 2-cyanoethyl N,N,N′,N′-tetraisopropylphosphordiamidite of formula (IV) (2.1 eq) were dissolved in anhydrous methylene chloride (0.4 M) at room temperature. 1H-tetrazole (2.1 eq) was added in four portions over 20 min and the reaction mixture stirred at room temperature for 1 h. The reaction mixture was cooled to 0° C., oxoadenine of formula (II) (1.0 eq) and imidazolium triflate (1.5 eq) were added, and the reaction mixture allowed to warm up to room temperature. The reaction was usually completed after 1 h at room temperature. The resulting phosphite can be purified at this stage (after reducing the volume by concentration under vacuum) or subsequently oxidized by addition of t-butyl hydroperoxide (1.5 eq) to the reaction mixture and stirring at room temperature for 30 min. After completion of the oxidation, the reaction mixture was concentrated under vacuum and purified by chromatography on silica gel. The resulting protected phosphotriester was dissolved in acetonitrile (0.06 M). Triethylamine (acetonitrile:TEA 1:0.35 v:v) was added and the reaction mixture stirred at room temperature for 6 to 18 h. Once the deprotection was complete, the reaction mixture was filtered over a Buchner filter and the isolated solid rinsed with acetonitrile and dried under high vacuum, or purified by chromatography on silica gel.

The characterisation data obtained were as expected.

Biological Testing of Compounds

### Example 1—HEK293 Assay TLR7 and TLR8 Activities

Method

Determination of TLR agonist activity was performed using the HEK293 binding assay.

This assay measures TLR7 and TLR8 selectivity and potency of the compounds tested. HEK293 cells expressing human TLR7 or TLR8 and NFKB responsive Secreted Embryonic Alkaline Phosphatase (SEAP) reporter gene were obtained from InvitroGen (San Diego, Calif.). These cells were maintained in culture media of Dulbecco's Modified Eagle Medium (DMEM) (Invitrogen, Grand Island, N.Y.), 10% Fetal Bovine Serum (FBS) (Sigma, St. Louis, Mo.) and selection antibiotics (Invitrogen, and Invitrogen). HEK293 stably transfected with human TLR7 (hTLR7) or human TLR8 (hTLR8) were stimulated for 24 h with aqueous formulations of compounds and culture supernatants were analysed for NFKB activation using the colorimetric SEAP detection kit QuantBlue (InvitroGen).

Choline salts of the compounds were prepared in aqueous solution (2% glycerol) and sonicated to reduce particle size to 120-370 nm.

Results

| | TLR7 ED₅₀ | TLR8 ED₅₀ | |
|---|---|---|---|
| Compound | (um) | (um) | TLR7/8 ratio |
| 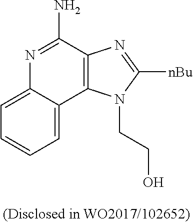 | 0.58 | 0.19 | 3.1 |
| 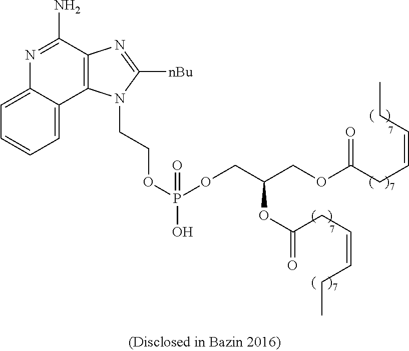 | 34.58 | 25.99 | 1.3 |
| 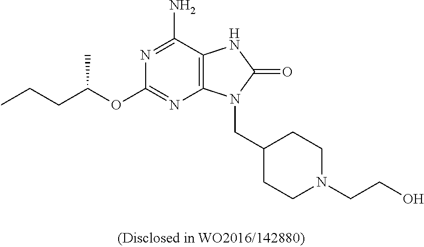 [SMILES: CCC[C@H](C)Oc1nc(N)c2[nH]c(=O)n(CC3CCN(CCO)CC3)c2n1] | 0.34 | 0.53 | 0.6 |
| 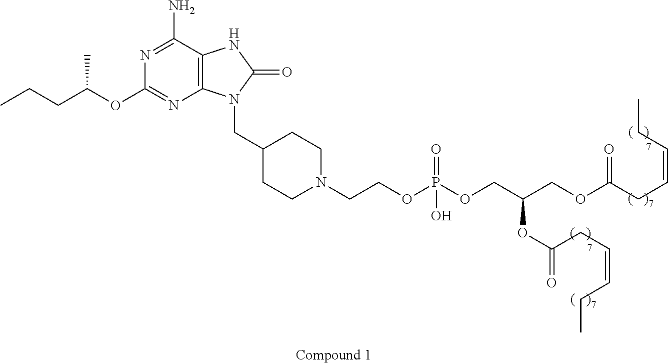 | 0.32 | 0.47 | 0.7 |

In this experiment the lipidated oxoadenine Compound 1 displayed a similar hTLR7 and hTLR8 activity to that of its parent core oxoadenine compound, however the lipidated compound was slightly more potent for both TLR7 and TLR8 activity.

Interestingly, as shown in the first two compound rows, there was a notable decrease in potency upon lipidation of the core imidazoquinoline structure. In contrast, equivalent lipidation of the oxoadenine compound did not result in a decrease in the potency.

Compound 1 was found to demonstrate a TLR7 bias and a high potency relative to similar compounds from the prior art.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. Embodiments are envisaged as being independently, fully combinable with one another where appropriate to the circumstances to form further embodiments of the invention. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### BIBLIOGRAPHY

Bazin et al., Bioorg. Med. Chem. Lett., 2015, 25:1318-1324
Bazin et al., Tetrahedron Letters, 2016, 57(19)
Berge et al., J. Pharm. Sci., 1977, 66:1-19
Gege et al., Chem Eur J, 2000, 6:111
Gerster et al., J. Med. Chem., 2005, 48:3481
Heil, F. Eur J Immunol., 2003, 33:2987
Hemmi, Nat Immunol., 2002, 3(2):196-200
Izumi et al., Bioorg Med Chem, 2003, 11:2541
Modica et al., ChemBioChem, 2012, 13:2331
Smith et al., Vaccine 2016 34(36):4304-12
Tashiro et al., Bioorg Med Chem, 2013, 21:3066

1. A compound of formula (I): 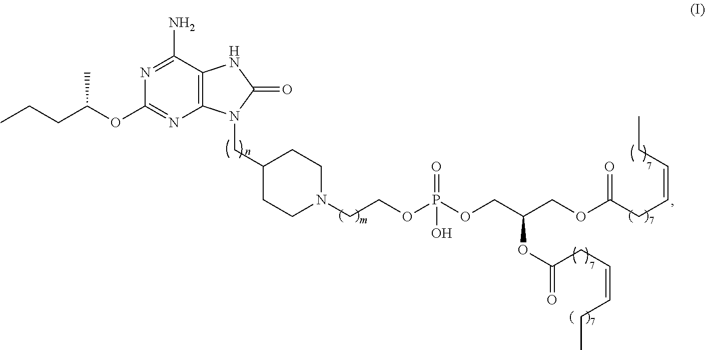
wherein n is 1, 2, 3, 4, or 5 and m is 0, 1, 2, 3, or 4
or a pharmaceutically acceptable salt and/or solvate and/or prodrug thereof.

2.-32. (canceled)

33. The compound according to claim 1, wherein n is 1.

34. The compound according to claim 1, wherein m is 1.

35. The compound according to claim 1, which is: 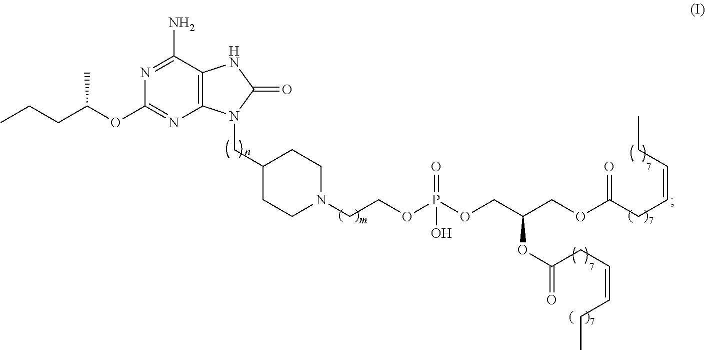
or a pharmaceutically acceptable salt and/or solvate thereof.

36. The compound according to claim 1, which is: 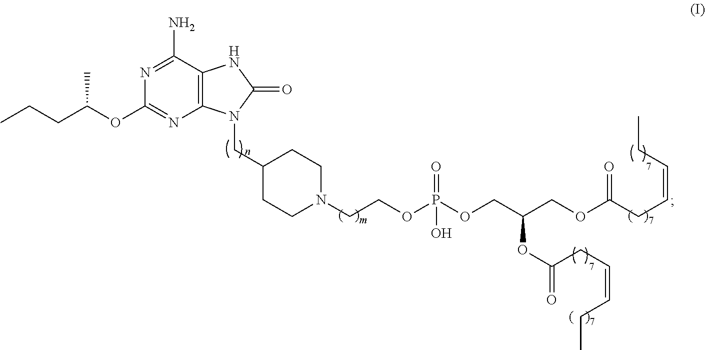
or a pharmaceutically acceptable salt thereof.

37. The compound according to claim 1, which is:

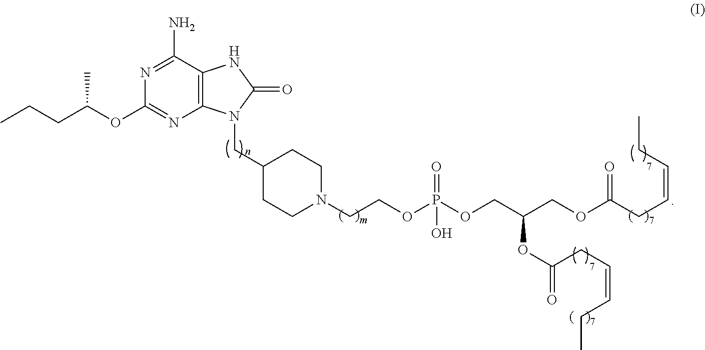

38. A method for eliciting an immune response in a subject, which comprises administering an effective amount of a compound of formula (I): 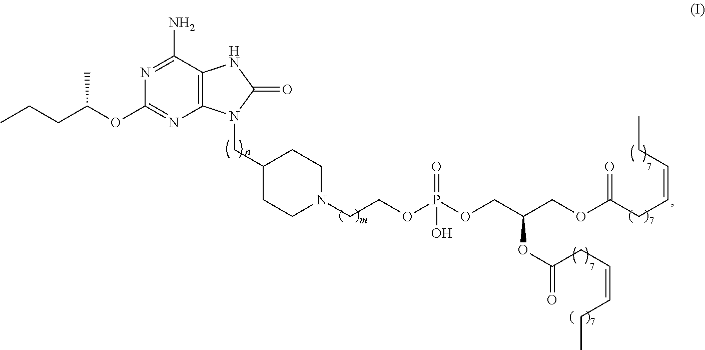
wherein n is 1, 2, 3, 4, or 5 and m is 0, 1, 2, 3, or 4, or a pharmaceutically acceptable salt and/or solvate and/or prodrug thereof, to the subject.

39. The method according to claim 38 wherein the subject is a human subject.

40. The method according to claim 38, wherein the compound of formula (I) is: 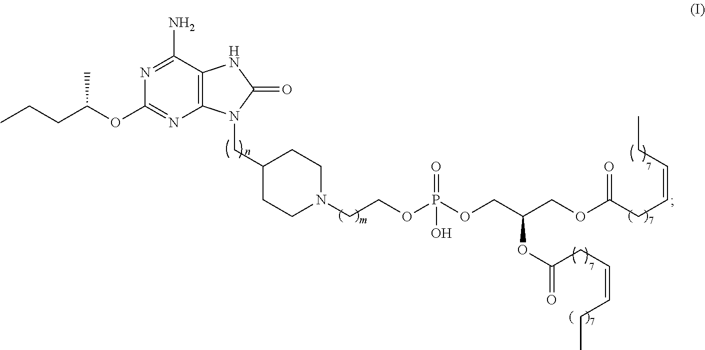
or a pharmaceutically acceptable salt and/or solvate thereof.

41. The method according to claim 38, wherein the compound of formula (I) is: 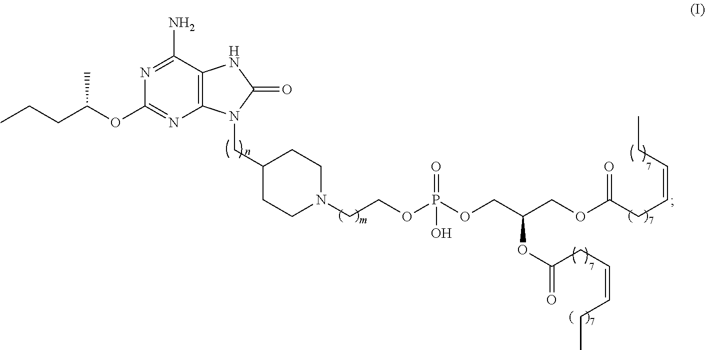
or a pharmaceutically acceptable salt thereof.

42. An immunogenic composition comprising a compound of formula (I): 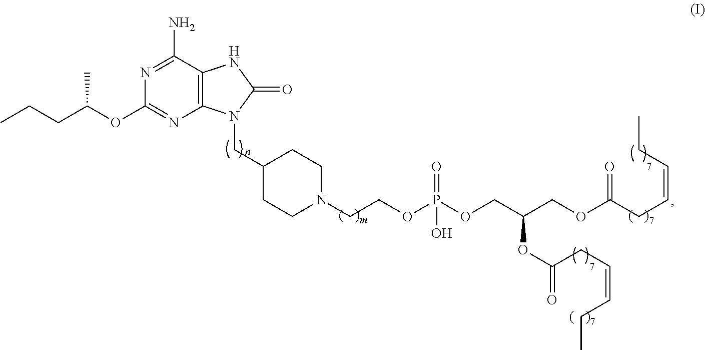
wherein n is 1, 2, 3, 4, or 5 and m is 0, 1, 2, 3, or 4, or a pharmaceutically acceptable salt and/or solvate and/or prodrug thereof and at least one immunogen or antigen.

43. The immunogenic composition according to claim 42, wherein the compound of formula (I) is: 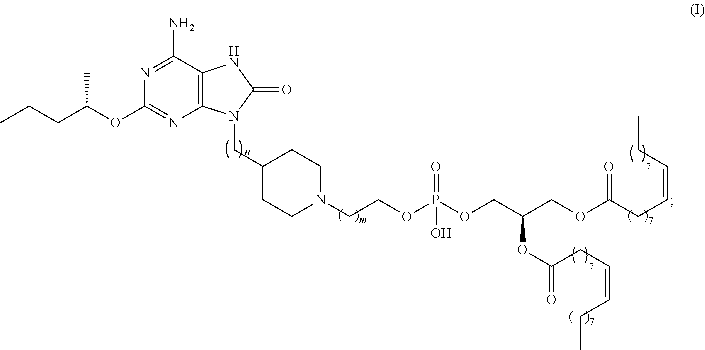
or a pharmaceutically acceptable salt and/or solvate thereof.

44. The immunogenic composition according to claim 42, wherein the compound of formula (I) is: 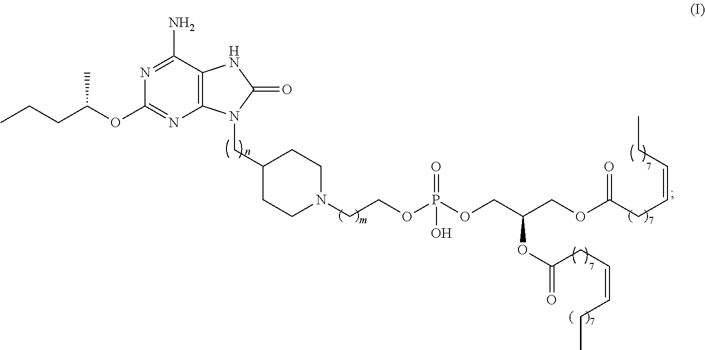
or a pharmaceutically acceptable salt thereof.

45. The immunogenic composition according to claim 42 wherein the antigen is derived from a human or non-human pathogen or from a cancer cell or tumor cell.

46. The immunogenic composition according to claim 42 wherein the antigen is derived from a human pathogen selected from bacteria, virus, fungi, parasitic microorganisms and multicellular parasites.

47. The immunogenic composition according to claim 42 wherein the composition is provided in a liposomal formulation.

48. The immunogenic composition according to claim 42 wherein the composition is provided in an aqueous formulation.

49. The immunogenic composition according to claim 42 wherein the composition has a pH of between 4 and 9.

50. The immunogenic composition according to claim 42 wherein the composition has an osmolality in the range of 250 to 750 mOsm/kg.

51. A compound of formula (II): 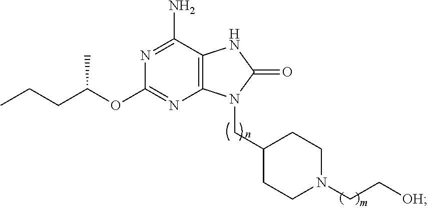
wherein n is 1, 2, 3, 4, or 5 and m is 0, 1, 2, 3, or 4.
